(19) United States
(12) Patent Application Publication
(10) Pub. No.: US 2009/0035402 A1
(43) Pub. Date: Feb. 5, 2009

(54) Method for Extracting Plants of the Genus Pelargonium, Extract Produced According to Said Method, and Use Thereof

(75) Inventor: Wilfried Kohnen, Haren (Ems) (DE)

Correspondence Address: MILLER, MATTHIAS & HULL, ONE NORTH FRANKLIN STREET, SUITE 2350, CHICAGO, IL, 60606, US

(73) Assignee: EMSPHARM GMBH, Haren (Ems) (DE)

(21) Appl. No.: 12/015,282

(22) Filed: Jan. 16, 2008

(30) Foreign Application Priority Data
Jul. 19, 2005 (DE) 102005034227 4-41

(51) Int. Cl. A61K 36/185; A61P 29/00; A61P 31/18; A61P 31/00

(52) U.S. Cl. 424/773; 424/725

(57) ABSTRACT

The present invention relates to a process for the extraction of plants, especially roots, of the plant genus Pelargonium, characterised in that the plants are extracted with an ethanol-free solvent or a mixture of ethanol-free solvents, selected from the group consisting of water and at least one monohydric alcohol with at least three carbon atoms, water and at least one polyhydric alcohol, water and at least one inorganic, organic, monoprotic or polyprotic acid or a derivative thereof, at least one vegetable oil and carbon dioxide.

The present invention relates to a process for the extraction of plants, preferably roots, of the plant genus Pelargonium and to an extract produced in particular according to the process, and also to the use thereof.

Pelargonium reniforme/sidoides is a plant which is traditionally used in folk medicine in southern Africa to treat gastrointestinal disorders and respiratory diseases. The plant was brought to England in 1897, and the plant and extracts were taken to Switzerland by Sechehaye in the Twenties. Extracts were used above all in homoeopathic preparations in tuberculosis therapy (Kolodziej, H., Kayser, O., Z. Phytotherap 19, 141-151, 1998).

It was possible to demonstrate in scientific studies that ethanolic extracts have an antimicrobial effect on some Gram-positive and Gram-negative bacteria (Kayser, O., Kolodziej, H., Planta Med. 63, 508-510, 1997) and possess immunomodulatory effects (Kayser, O., Kolodziej, H., Kiderlen, A. F., Radtke, O., Phytomedicine 2003; 10 Suppl 4:18-24.

The main ingredients of Pelargonium sidoides roots are proanthocyanidines, oligomeric hydrolysable tannins, coumarins, flavonoids and caffeic acid derivatives (Kayser, O., Dissertation, Free University of Berlin, 1997). A characteristic feature is the presence of in some cases highly oxygenated coumarins, which are thought to be partially responsible for the biological effect (Kayser, O., Kolodziej, H., Phytochemistry, 39, 1181-1185, 1995).

In the past, extracts of the root material of Pelargonium sidoides and Pelargonium reniforme, and also of other plants of the Pelargonium genus were produced by maceration and/or percolation, using pure water, mixtures of water and an organic solvent such as acetone (Kayser, O., Kolodziej, H., Phytochemistry, 39, 1181-1185, 1995), methanol (Bladt, S., Wagner; H., Dtsch. Apoth.-Ztg., 128, 292-296, 1988) or ethanol (WO 03/028746 A1). In the meantime, Pelargonium lobatum and Pelargonium triste are also known members of the plant genus Pelargonium. There is an ethanolic extract of Pelargonium reniforme/sidoides stabilised with glycerol which is commercially available and is used in therapeutic applications (Rote Liste 2006). Compared to extraction with water, methanol or acetone, ethanolic extraction achieves improved yields of extracts, and extraction is possible at room temperature. However, the use of ethanolic extracts of P. reniforme/sidoides is considered problematic in specialist circles, since children are the preferred group of patients who take extracts used for medical purposes. This problem also applies to patients who should not or must not take ethanol, for medical reasons, such as alcoholics, epileptics or Parkinson's patients.

The desire for ethanol-free liquids from plants in other pharmaceutical fields has led in the past to the development of methods of preparation without ethanol, which, in contrast to fresh plant juices, infusions and aqueous tonics now also contain lipophilic plant ingredients. Examples of the solution and extraction media used have been polyethylene glycol, glycerol, sorbitol or propylene glycol, alone or in combination with water. In a dissertation by Vogel, K., Dissertation, Tübingen University, 1992, it was shown that the lipophilic ingredients of chamomile, namely chamazulene and bisabolol, are not only readily soluble in propylene glycol, but can also be stabilised chemically and physically. Chamomile belongs to a different plant genus from Pelargonium. In chamomile extraction, however, it was found that, by using the polyhydric alcohol propylene glycol, predominantly only lipophilic components were extracted, whereas hardly any hydrophilic components were obtained (Vogel, K., Dissertation, Tübingen University, 1992). Compared to ethanol-free extracts, ethanol/water mixtures possess certain technological and microbiocidal advantages; in particular, a greater yield of extract can be expected. At present, a propylene glycol/glycerol/water mixture is used in order, in some finished medicinal preparations, to dissolve the ethanolic viscous or acetonic extract (e.g. Tebonin forte solution) Sorbitol/water mixtures are used in order to offer the drug Prospan, which is used in paediatrics (Rote Liste 2006).

The present invention is based on the problem of improving the generic process in such a way that the disadvantages of the state of the art are overcome. In particular, the object is to provide a process which enables an ethanol-free extraction of plants, especially roots, of the plant genus Pelargonium, in order to make it possible to administer the product to patients who should not or must not take ethanol, for medical reasons (such as children, alcoholics, epileptics or Parkinson's patients). The process of the invention is further intended to lead to an extract which is substantially equivalent to the extract obtained by water/ethanol extraction. The extraction agents used should be toxicologically harmless. In particular, the process is intended to provide an extract which is qualitatively and quantitatively similar to the aqueous ethanolic extracts.

In addition, it is an object of the present invention to provide an extract, especially as prepared in accordance with the process, and to enable its use.

The first problem is solved in that the plants are extracted with an ethanol-free solvent or a mixture of ethanol-free solvents which is or are selected from water and at least one monohydric alcohol with at least three carbon atoms, water and at least one polyhydric alcohol, water and at least one inorganic, organic, monoprotic or polyprotic acid or a derivative thereof, at least one vegetable oil and carbon dioxide. Sugar and sugar alcohols are preferably also understood to be polyhydric alcohols.

It is preferable in this connection that the extraction should comprise percolation and/or single-step or multi-step, preferably two-step maceration.

It is particularly preferable that the polyhydric alcohol should be selected from the group consisting of polyethylene glycol, glycerol, propylene glycol, sorbitol, xylitol or mixtures thereof.

It is proposed here that the polyethylene glycol should have a molecular weight of from about 40 to about 40,000, preferably of from about 200 to about 2,000, particularly preferably of from about 200 to about 800.

In one embodiment, the plants are chopped, ground and/or dried and/or are used fresh.

It is particularly preferable that the weight ratio of water:polyhydric alcohol should lie within the range from 95:5 to about 5:95.

It is also proposed that the weight ratio of water:organic monoprotic or polyprotic acid should lie within the range from 99.9:0.1 to about 80:20.

Alternatively, it is proposed that at least one vegetable oil or carbon dioxide should be used as the extraction agent.

It is likewise proposed as an alternative that the weight ratio water:inorganic acid should lie within the range from 99.9:0.1 to about 80:20. The inorganic acid is preferably phosphoric acid or its alkali or earth alkali salts.

It is preferred that the weight ratio of water:polyhydric alcohol in the case of maceration should range from about 80:20 to about 60:40, preferably about 70:30, and that the weight ratio of water:polyhydric alcohol in the case percolation should range from about 90:10 to about 70:30, preferably about 80:20.

It can also be provided that the plants should be mashed before percolation.

The second problem is solved by an extract the plant genus Pelargonium, dissolved in an ethanol-free solvent or a mixture ethanol-free solvents, selected from the group consisting of water and at least one monohydric alcohol with at least three carbon atoms, water and at least one polyhydric alcohol, water and at least one inorganic, organic, monoprotic or polyprotic acid or a derivative thereof, at least one vegetable oil and carbon dioxide.

It is most preferred that the extract should be obtained from roots of the plant Pelargonium sidoides, Pelargonium reniforme, Pelargonium lobatum and/or Pelargonium triste.

In one embodiment, it is also proposed that the extract should contain a thickening agent in addition and should be present in a solid, hardened form.

In this case, the extract can be present in the form a sweet to suck, a lozenge to suck, a buccal or lingual dosage form or a lollipop.

Finally, the use of the extract of the invention for the treatment of acute and/or chronic inflammatory diseases and/or infections in mammals is proposed.

Use in human beings, pets and domestic animals, preferably horses, dogs, cats, pigs or chickens, is preferred.

It is also proposed that the root extract should be administered orally.

The extract can preferably also be used for the treatment of or prophylaxis against HIV infections or HIV-associated infections HIV-associated infections of this kind may be bacterial, other viral, fungal and/or parasitic infections. More specific examples are listed in DE 10 2004 032 439 A1.

It has surprisingly been found that, with the process of the invention, an extract, preferably a root extract, of the plant genus Pelargonium can be obtained in the same yield and quality as with extraction on a water/ethanol basis. At the same time, it has been found that the known antimicrobial and immunostimulating effects are achieved with the process of the invention. With the process of the invention, large proportions of lipophilic and hydrophilic ingredients can be extracted. Since the process of the invention can be operated ethanol-free, the extract obtained can safely be used for the treatment of children, alcoholics, epileptics and Parkinson's patients.

In the process the invention, the extraction is preferably carried out in the form percolation and/or two-step maceration.

Percolation is preferably carried out with different mixtures water and polyhydric alcohol. One preferred polyhydric alcohol is sorbitol, for example in a mixture water/glycerol/sorbitol 70:15:15 (% by weight), or polyethylene glycol, for example in a mixture of water/polyethylene glycol/glycerol 70:20:10 (% by weight). Percolation can, however, also be carried out by mashing, with the mashing and subsequent percolation carried out with water/alcohol mixtures the same or different concentration. An advantage that appeared here was that mashing with a water/alcohol ratio 70:30 (% by weight) and percolation with a water/alcohol ratio 80:20 (% by weight) led to the best yields. A ratio of root mash to extraction medium of 20:80 (% by weight) results in a final concentration of the polyhydric alcohol(s) of approximately 22%. The extracts the invention contain a proportion coumarin, based on scopoletin, which is the same order magnitude as that the ethanolic extracts.

In extraction experiments with the ground and dried root of Pelargonium sidoides with mixtures of water and polyhydric alcohol, it was surprisingly found that, depending on the composition of the extraction medium, lipophilic substances such as coumarins, on the one hand, but also water-soluble phenols, on the other hand, can be extracted in the same extraction step. Against the background of the existing state of knowledge regarding Pelargonium sidoides and Pelargonium reniforme this could not have been expected. Both with percolation and with two-step maceration, which are described in greater detail below in the examples, it was possible to obtain quantities of extract which were qualitatively and quantitatively comparable to those of ethanolic extracts.

One particularly preferred embodiment the root extract obtained in accordance with the invention consists in adding to the extract a thickening agent, glucose syrup for example, in such a quantity that the extract solidifies, or hardens. Then it is possible to administer the hardened extract in the form of a sweet to suck, a lozenge to suck, a buccal or lingual dosage form or a lollipop, for example, which can facilitate the treatment of children in particular. In addition, the extract the invention can also be formulated and administered in a conventional liquid preparation, such as in the form drops, linctus, syrup, etc.

When the extracts of the invention were examined to determine their antimicrobial and immunostimulating effect, a pronounced effect was found on Gram-positive microbes, such as Staphylococcus aureus, Streptococcus pneumoniae and Gram-negative microbes, such as E. coli, Proteus mirabilis and Haemophilus influenzae. When they were examined for immunostimulation, an induction of γ-IFN and TNF alpha was found in murine macrophages infected with Leishmania donovani.

Possible extraction agents are certain monohydric or polyhydric alcohols and derivatives, such as aliphatic and aromatic monohydric or polyhydric alcohols, inorganic or organic, monoprotic or polyprotic acids and derivatives, vegetable oil and carbon dioxide. Preferred mixing ratios are stated above.

Examples of specific, particularly preferred extraction agents are:

Polyhydric alcohols, including sugar and sugar alcohols, polyethylene glycol (PEG) 100-4000, propylene glycol, polypropylene glycol 100-4000, butane diol in possible substitution forms and its derivatives, butyl alcohol, 1,3-butylene glycol, carboxymethyl cellulose and its derivatives, such as carboxymethyl cellulose, carboxyethyl cellulose, dextran, dextrin, dextrose, ethyl cellulose, ethylene glycol derivatives, such as ethylene glycol distearate, ethylene glycol monobutyl ether, ethylene glycol monoethyl ether, glucose, glycerine and its etherified and esterified derivatives, especially glycerine esterified with long-chain fatty acids or etherified with long-chain fatty alcohols, lactose, monoglycerides and diglycerides with their esters and alkali and earth alkali salts, octanol, polysorbate 20-80, polyvinyl acetate, polyvinyl alcohol, propylene glycol alginate, sorbitan and its monoesterified and polyesterified derivatives, sorbitol, xylose, xylitol, organic monoprotic and polyprotic acids and their alkali and earth alkali salts, such as ascorbic acid, tartaric acid, malic acid, heteropolysaccharides, monosaccharides, disaccharides, trisaccharides, tetrasaccharides, carbon dioxide, vegetable oils, lecithin and its derivatives, phosphoric acid and its alkali and earth alkali salts and polyoxyethylene, preferably esterified with saturated or unsaturated fatty acids, such as polysorbate 20-80.

Likewise preferred are aliphatic phenyl alcohols, such as isobutyl, propyl or ethylphenyl alcohol, methylbenzyl acetate, methylbenzyl butyrate and other acid derivatives, liquid, saturated and unsaturated fatty acids and fatty alcohols with chain lengths from C₆ to C₁₆, benzyl alcohol and its derivatives, isoamyl alcohol and its derivatives, isobutyl alcohol and its derivatives.

The invention will now be explained with reference to the following, non-limiting examples. In older to obtain comparable analytical and pharmacological data, the scopoletin of all the extracts was determined.

### EXAMPLE 1

100 g dried and ground root of Pelargonium sidoides were pre-steeped for 24 h with 200 g of a water/sorbitol mixture (70 g sorbitol in 100 ml water) and then percolated for 8 hours with 800 ml of the same water/sorbitol mixture. The raw extract was filtered through Seitz Supra 1500 and filled

TABLE 1
Amount of extracts obtained and correlated biological effect

| Batch | TP %¹ | TC %² | IFN³ | TNF⁴ | E. coli⁵ | S. a.⁶ | S. p.⁷ | P. m.⁸ | H. i.⁹ |
|---|---|---|---|---|---|---|---|---|---|
| Example 1 | 15.0 | 2.59 | 0.3 | 137 | 11 | 10 | 12 | 9 | 8 |

¹TP: Total phenol, shown in percent
²TC: Total coumarin, shown in percent
³IFN: γ-interferon, shown in EC₅₀ (μg/ml)
⁴TNF: Tumour necrosis factor alpha, shown in U/mL
⁵E. coli: Escherichia coli, shown as diameter of the inhibiting areola in mm
⁶S. a: Staphylococcus aureus, shown as diameter of the inhibiting areola in mm
⁷S. p.: Streptococcus pneumoniae, shown as diameter of the inhibiting areola in mm
⁸P. m.: Proteus mirabilis, shown as diameter of the inhibiting areola in mm
⁹H. i.: Haemophilus influenzae, shown as diameter of the inhibiting areola in mm

### EXAMPLE 2

100 g dried and ground root of Pelargonium sidoides were pre-steeped for 24 h with 200 g of a water/PEG400 mixture (water:PEG400=10:90 (% by weight)) and then percolated for 8 hours with 800 ml of the same water/PEG400 mixture. The raw extract was filtered through Seitz Supra 1500 and filled.

TABLE 2
Amount of extracts obtained and correlated biological effect

| Batch | TP %¹ | TC %² | IFN³ | TNF⁴ | E. coli⁵ | S. a.⁶ | S. p.⁷ | P. m.⁸ | H. i.⁹ |
|---|---|---|---|---|---|---|---|---|---|
| Example 2 | 16.3 | 3.31 | 0.5 | 149 | 14 | 14 | 11 | 10 | 9 |

### EXAMPLE 3

20 g each of dried and ground root drug of P. sidoides were pre-steeped for 16 h in various media formulations (A1-A15) (see Table 3) and then percolated with 160 g each of two different extraction media (E1, E2). The extraction medium E1 consisted of water/glycerol/sorbitol 80:10:10 (% by weight), and extraction medium E2 consisted of water/glycerol/PEG400 85:12.5:2.5 (% by weight). Percolation continued for 8 h, and the law extract was filtered though a flitted glass filter size G4. In addition to the ethanol-free extracts, an ethanol-containing extract (A16) was also prepared for comparative purposes, its preparation being in accordance with the details provided in Example 1 of WO 03/028746 A1.

TABLE 3
Formulation mixtures and extraction media (all details in % by weight)

| Formulation | Extraction | PEG400 | Soibitol | Propylene glycol | Glycerol | Water | Total |
|---|---|---|---|---|---|---|---|
| A1 | E1 | 0 | 10 | 0 | 20 | 70 | 100 |
| A2 | E1 | 0 | 15 | 0 | 15 | 70 | 100 |
| A3 | E1 | 0 | 20 | 0 | 10 | 70 | 100 |
| A4 | E1 | 0 | 10 | 0 | 10 | 80 | 100 |
| A5 | E1 | 0 | 10 | 10 | 10 | 70 | 100 |
| A6 | E2 | 20 | 0 | 0 | 10 | 70 | 100 |
| A7 | E2 | 15 | 0 | 0 | 15 | 70 | 100 |
| A8 | E2 | 10 | 0 | 0 | 20 | 70 | 100 |
| A9 | E2 | 5 | 0 | 0 | 25 | 70 | 100 |
| A10 | E2 | 20 | 0 | 5 | 5 | 70 | 100 |
| A11 | E2 | 15 | 5 | 0 | 10 | 70 | 100 |
| A12 | E2 | 10 | 5 | 5 | 10 | 70 | 100 |
| A13 | E2 | 5 | 10 | 0 | 10 | 75 | 100 |
| A14 | E2 | 10 | 0 | 10 | 10 | 70 | 100 |
| A15 | E2 | 5 | 10 | 0 | 15 | 70 | 100 |

TABLE 4
Amount of extracts obtained and correlated biological effect

| Formulation | TP %¹ | TC %² | IFN³ | TNF⁴ | E. coli⁵ | S. a.⁶ | S. p.⁷ | P. m.⁸ | H. i.⁹ |
|---|---|---|---|---|---|---|---|---|---|
| A1 | 15.3 | 2.64 | 0.4 | 145 | 10 | 10 | 11 | 10 | 11 |
| A2 | 15.3 | 2.59 | 2.3 | 132 | 8 | 9 | 10 | 9 | 9 |
| A3 | 16.6 | 2.12 | 2.1 | 144 | 10 | 11 | 12 | 10 | 10 |
| A4 | 16.8 | 2.54 | 1.7 | 156 | 8 | 9 | 10 | 11 | 11 |
| A5 | 15.3 | 2.38 | 1.9 | 123 | 12 | 11 | 11 | 10 | 10 |
| A6 | 18.5 | 3.29 | 3.2 | 198 | 18 | 16 | 15 | 17 | 18 |
| A7 | 16.1 | 3.26 | 3.6 | 176 | 14 | 14 | 13 | 12 | 14 |
| A8 | 16.4 | 3.31 | 3.1 | 173 | 12 | 14 | 12 | 13 | 12 |
| A9 | 17.8 | 2.77 | 3.4 | 182 | 12 | 13 | 15 | 14 | 14 |
| A10 | 16.5 | 3.61 | 3.7 | 203 | 18 | 16 | 15 | 18 | 16 |
| A11 | 17.3 | 3.25 | 3.5 | 183 | 10 | 9 | 11 | 11 | 12 |
| A12 | 16.8 | 3.13 | 3.3 | 175 | 12 | 10 | 11 | 12 | 11 |
| A13 | 16.9 | 2.89 | 3.2 | 182 | 12 | 11 | 12 | 10 | 10 |
| A14 | 15.6 | 2.78 | 3.0 | 175 | 10 | 9 | 11 | 9 | 10 |
| A15 | 15.2 | 2.93 | 3.2 | 177 | 12 | 9 | 11 | 13 | 12 |
| A16 | 16.5 | 3.25 | 3.3 | 192 | 10 | 10 | 12 | 11 | 11 |

### EXAMPLE 4

Compared to percolation, a modified form maceration in accordance with the DAB 10 regulation (German Pharmacopoeia) was carried out. 20 g of the ground and dried root of P. sidoides were suspended with 140 ml of a water/sorbitol mixture (70 g sorbitol in 100 ml Aqua dest) and allowed to stand for 5 days with occasional shaking. After that, the mixture was filtered, and the raw extract trapped. The moist drug material was then macerated a second time in the same way. The extract solution was likewise trapped and combined with the first raw extract.

TABLE 5
Amount of extracts obtained and correlated biological effect

| Formulation | TP %¹ | TC %² | IFN³ | TNF⁴ | E. coli⁵ | S. a.⁶ | S. p.⁷ | P. m.⁸ | H. i.⁹ |
|---|---|---|---|---|---|---|---|---|---|
| Example 4 | 12.1 | 2.11 | 0.3 | 112 | 9 | 8 | 8 | 7 | 6 |

### EXAMPLE 5

Compared to percolation, a modified form of maceration in accordance with the DAB 10 regulation was carried out. 20 g the ground and dried root were suspended with 140 ml water/PEG400 mixture (water:PEG400=10:90 (% by weight)) and allowed to stand for 5 days with occasional shaking. After that, the mixture was filtered, and the taw extract trapped. The moist drug material was then macerated a second time in the same way. The extract solution was likewise trapped and combined with the first raw extract

TABLE 6
Amount of extracts obtained and correlated biological effect

| Formulation | TP %¹ | TC %² | IFN³ | TNF⁴ | E. coli⁵ | S. a.⁶ | S. p.⁷ | P. m.⁸ | H. i.⁹ |
|---|---|---|---|---|---|---|---|---|---|
| Example 5 | 15.5 | 3.21 | 0.6 | 156 | 14 | 16 | 14 | 14 | 12 |

### Analytical and Pharmacological Methods

### A. Determining the Total Phenols

The proportion of total phenols was determined in accordance with the method specified in the German Pharmacopoeia for tannins (DAB 2002). The amount was determined photometrically after reacting with molybdate/tungstate reagent. For this purpose, the raw extract was taken directly, alkalised with sodium carbonate solution and then mixed with molybdate/tungstate reagent. After centrifugation, the extinction of the supernatant solution at 720 nm was measured against water. The results found against extraction medium E1 or E2 did not exhibit any difference compared to water. The results were calculated on the basis of the epicatechin.

### B. Determining the Total Coumarins

The total coumarins were determined using HPLC over an RP-18 column. The mobile phase used was an acetonitrile/water/phosphonic acid gradient (10:990:4 to 205:795:4). Detection was per formed at 330 nm. The individual coumarin peaks were calculated as scopoletin.

### C. Determining the Antimicrobial Effect

50 μl each of the extracts of the invention were diluted to 200 μl in 150 ml Muellr-Hinton medium, and the antimicrobial effect was determined in accordance with DIN 58 940, 1995, and the method described by Van den Berghe D. A., Vlietnick, A. J., in: Methods in Plant Biochemistry, Assays for Bioactivity, Vol. 6, Dey, P. M., Harborne, J. B. (eds.), Academic Press, London, San Diego, New York, Boston, Sydney, Tokyo, Toronto, 47-99 (1991). The effect was determined by measuring the inhibiting areola on agar plates, into which wells were punched before inoculation. The reference strains were obtained from the German collection of strains for micro-organisms and cell cultures (DSMZ) in Brunswick: Escherichia coli ATCC 25922, Haemophilus influenzae ATCC 33379. All the other strains were obtained as an in-house strain from patients' material at the University Medicinal Centre UMCG, Groningen, NL.

### D. Determining the Immunomodulating Effect

### Testing for TNF Synthesis and Release from Murine L-929 (TNF) and Human WEHI Fibroblasts

Cells of the murine fibroblast cell line L-929 (TNF) No. 570.7 and WEHI 164/E clone 13 No 5676 were precultured in R5 medium at 37° C., 6% CO₂. The culture medium in the nutrient flask was discarded, and the adherent cells in the flask were rinsed with 10 mL HBSS (Henk balanced salt solution) (without FCS (foetal calf serum)). The HBSS solution was likewise discarded, 5 mL trypsin/EDTA solution were added to the flask and incubated for 15 minutes at 37° C., 6% CO₂. The enzymatic detachment of the cells was stopped by adding 5 mL R5 medium, and the cell suspension was collected in Falcon tubes. In order to harvest the cells, the suspension was centrifuged twice at 1,100 r.p.m. for 12 minutes at room temperature and washed three times with 10 mL HBSS solution. The cell count was per formed according to the Neubauer method (3 97×10⁷ for L-929 (TNF) and 13×10⁷ for WEHI 164/E), which were in each case adjusted with R5 solution to 3×10⁴ cells/well in 100 μL (corresponding to 3×10⁵ cells/mL). The standardised cell suspension was plated on microtitre plates (96 wells). For control purposes, medium controls (cell-free) were pipetted into two wells, negative controls (cells with R5 medium) were pipetted into two further wells, and a positive control (cells in R5 solution with 5 μL of a TNF solution adjusted to 10 U/mL) into one well. The plated microtitre plates were incubated over night at 37° C., 6% CO₂.

Once the cell suspensions had been incubated over night, the supernatants were drawn off the cells adhering to the bottom and adjusted with differently diluted actinomycin D solutions and test solutions. The activated microtitre plates were incubated for 18 hours at 37° C., 6% CO₂. After incubation, the positive control was examined microscopically for sufficient cell lysis. The medium from the microtitre plate was discarded and the plate tapped dry on a mat. 100 μL a 0.5% crystal violet solution were placed in each well and stained for 3 minutes. After that, the staining solution was discarded, each microtitre plate was washed four times in distilled water, tapped dry on a mat and dried at 50° C. for 30 minutes. 100 μL 33% acetic acid were then placed in each well, the microtitre plates were shaken for 10 minutes and the absorption at 592 nm measured in the ELISA device.

### Determining the Cytoprotective Effect by Obtaining the Values for IFN in the EMC Virus/L929 (IFN) Test Model

L929 (IFN) cells (batch no. 5577) were cultured, trypsinized, washed and counted in 100-mL culture dishes. The suspension was adjusted to 2×10⁵ cells/mL and sown in microtitre plates with 2×10⁴ cells/well, which were incubated over night at 37° C., 5% CO₂. After that, the supernatant was replaced by 100 μL each medium control, cell control, virus control with rMuIFN-γ control, rMuIFN-γ standard (100 U/mL) and test solution, all of which were diluted lineally.

The samples were incubated for exactly eight hours at 37° C., 5% CO₂. After the end of the incubation time, 100 μL each of the vital suspension used were pipetted into the vital and interferon controls and into the test solutions and again incubated over night at 37° C., 5% CO₂.

After 22 hours, fully developed viral activity appeared (cell lysis must be distinctly pronounced in the vital control), so that the lysis was interrupted. The supernatants were discarded via the wash basin 100 μl of a 0.5% crystal violet solution were pipetted into each well. After three minutes' staining time, the surplus solution was removed and the microtitre plates rinsed with distilled water and dried for 10 minutes at 50° C. 100 μL acetic acid (33%) were pipetted into the completely dry wells and shaken for 10 minutes. The absorption of the crystal violet staining was determined on the ELISA reader at 550 nm. The average absorption the rMuIFN-γ control was set at 100% cell protection and that of the viral control at 0%. The ED₅₀ value of the rMuIFN-γ standard was at 1,076 U/mL (determined from 8 standard curves).

The features of the invention disclosed in the above description and in the claims can be essential to implementing the invention in its various embodiments both individually and in any combination.

1. A method for extracting plants, especially roots, of a plant genus Pelargonium, comprising extracting the plants with:
an ethanol-free solvent or a mixture of ethanol-free solvents selected from the group consisting of water and at least one monohydric alcohol with at least three carbon atoms, water and at least one polyhydric alcohol, water and at least one inorganic, organic, monoprotic or polyprotic acid or a derivative thereof;
at least one vegetable oil; and
carbon dioxide.

2. The method of claim 1, in which the extracting comprises percolation and/or single-step or multi-step, preferably two-step, maceration.

3. The method of claim 1, in which the polyhydric alcohol is selected from a group consisting of polyethylene glycol, glycerol, propylene glycol, sorbitol, xylitol or mixtures thereof.

4. The method of claim 3, in which the polyethylene glycol has a molecular weight of from about 40 to about 40,000, preferably from about 200 to about 2,000, particularly preferably from about 200 to about 800.

5. The method of claim 1, in which the plants are chopped, ground and/or dried and/or are used fresh.

6. The method of claim 1, in which a weight ratio of water:polyhydric alcohol lies within a range from 95:5 to about 5:95.

7. The method of claim 1, in which a weight ratio of water:organic monoprotic or polyprotic acid lies within a range from 99.9:0.1 to about 80:20.

8. The method of claim 1, in which at least one vegetable oil or carbon dioxide is used as an extraction agent.

9. The method of claim 1, in which a weight ratio of water:inorganic acid lies within a range from 99.9:0.1 to about 80:20.

10. The method of claim 6, in which a weight ratio of water:polyhydric alcohol during maceration lies within a range from 80:20 to about 60:40, preferably about 70:30.

11. The method of claim 6, in which a weight ratio of water:polyhydric alcohol during percolation lies within a range from about 90:10 to about 70:30, preferably about 80:20.

12. The method of claim 2, in which the plants are mashed before percolation.

13. An extract of the plant genus Pelargonium, dissolved in an ethanol-free solvent or a mixture of ethanol-free solvents, selected from the group consisting of water and at least one monohydric alcohol with at least three carbon atoms, water and at least one polyhydric alcohol, water and at least one inorganic, organic, monoprotic or polyprotic acid or a derivative thereof, at least one vegetable oil, and carbon dioxide.

14. The extract of claim 13, in which the extract is obtained from roots of the plant Pelargonium sidoides, Pelargonium reniforme, Pelargonium lobatum and/or Pelargonium triste.

15. The extract of claim 13, further comprising a thickening agent in a solid, hardened form.

16. The extract of claim 15, in which the extract is present in the form of a sweet to suck, a lozenge to suck, a buccal or lingual dosage form, or a lollipop.

17. Use of the extract as claimed in claim 13 for the treatment of acute and/or chronic inflammatory diseases and/or infections in mammals.

18. The use of claim 17 in human beings, pets and domestic animals, preferably horses, dogs, cats, pigs or chickens.

19. The use of claim 17, characterised in which the extract is administered orally.

20. The use of claim 17 for treating or prophylaxis against HIV infections or HIV-associated infections.
